(19) United States
(12) Patent Application Publication
(10) Pub. No.: US 2004/0024048 A1
(43) Pub. Date: Feb. 5, 2004

(54) Medicaments for treating dementia

(76) Inventors: Klaus Wessel, Bad Vilbel (DE); Gerald Munch, Leipzig (DE); Klaus Hager, Hannover (DE); Marlene Kenklies, Hannover (DE); Michael Lobisch, Reichelsheim/Blofeld (DE); Manfred Peukert, Oberursel/Ts (DE); Harald Borbe, Mainz (DE); Andreas Marahrens, Isernhagen (DE)

Correspondence Address: BURNS DOANE SWECKER & MATHIS L L P, POST OFFICE BOX 1404, ALEXANDRIA, VA, 22313-1404, US

(21) Appl. No.: 10/297,481

(22) PCT Filed: Jun. 7, 2001
(86) PCT No.: PCT/EP01/06478

(30) Foreign Application Priority Data
Jun. 8, 2000 (DE) 100 27 968.6

(51) Int. Cl. A61K 31/714; A61K 31/60; A61K 31/498; A61K 31/525; A61K 31/51; A61K 31/045; A61K 35/78

(52) U.S. Cl. 514/440; 424/730; 424/754; 514/458; 514/393; 514/52; 514/276; 514/251; 514/250; 514/561; 514/553; 514/165; 514/560; 514/355; 514/729; 514/690; 424/702

(57) ABSTRACT

The invention relates to the use of α-lipoic acid in reduced or oxidized form or derivatives thereof with intact dithiolane structure in the form of enantiomers or pharmaceutically acceptable salts, amides, esters, thioesters, ethers or metabolites for the adjuvant therapy of dementias.

The invention relates to the use of α-lipoic acid in reduced or oxidized form or derivatives thereof with intact dithiolane structure in the form of enantiomers or pharmaceutically acceptable salts, amides, esters, thioesters, ethers or metabolites for the adjuvant therapy of dementias.

The prior art is set forth in the following publications:

DE 4343592 C2, EP 0382066 A2, DE 4439477 C2, EP 0530446B1, EP 0855396 A1, WO 99/06040, U.S. Pat. No. 5,532,269, DE 4343593 A, U.S. Pat. No. 5,599,835, U.S. Pat. No. 5,569,670, EP 0869126 A1, PCT/GB98/02155, PCT/US99/11178, DE 4327462 Al,

The DANA Consortium, A randomized, double-blind placebo-controlled trial of deprenyl and thioctic acid in human immunodeficiency virus-associated cognitive impairement. Neurology 50: 645-651; 1998,

Di Ricco A, Roesler R, Quevedo J, Walz R, Bianchin M. A randomized, double-blind placebo-controlled trial of deprenyl and thioctic acid in human immunodeficiency virus-associated cognitive impairement. Neurology 52: 1920, 1999,

Boysen K H. Erfahrungen mit dem Präparat Thioctacid auf einer psychiatrischen Station. Med. Welt; 395-400, 1967,

Tirosh O. Sen C K, Roy S, Kobayashi S, Packer L. Neuroprotective effects of α-lipoic acid and ist positively charged amide analogue. Free Rad Biol Med 26 (11/12), 1418-1426, 1999,

Packer L, Tritschler HJ, Wessel K. Neuroprotection by the metabolic antioxidant α-lipoic acid. Free rad Biol Med, 22 (1/2), 359-378, 1997,

Stoll S, Hartmann H, Cohen S A, Muller W E. The potent free radical scavenger α-lipoic acid improves memory in aged mice: putative relationship to NMDA receptor deficits. Pharmacol Biochem Behav 46, 799-805, 1993,

Stoll S, Rostock A, Bartsch R, Korn E, Meichelbock A, Muller W E. The potent free radical scavenger α-lipoic acid improves memory in rodents. Ann NY Acad Sci 717, 122-128, 1994,

Loske C, Neumann A, Cunningham A M, Nichol K, Schinzel R, Riederer P, Münch G. Cytotoxicity of advanced glycation endproducts is mediated by oxidative stress.

J Neural Transm, 219-229, 1998, Hagen T M, Russel T, Ingersoll T, Lykkesfeldt J, Liu J, Wehr C M, Vinarsky V, Batholomew J C, Ames B N. R(+)-alpha-lipoic acid supplemented old rats have improved mitochondrial function, decreased oxidative damage and increased metabolic rate. FASEB J 13: 411-418, 1999 and Summary of product characteristics for Thioctacid, ASTA Medica 1999.

α-Lipoic acid (thioctic acid, 1,2-dithiolane-3-pentanoic acid) is a biological cofactor of α-keto-acid dehydrogenases in the mitochondria and is involved in the biooxidation of these acids (pyruvate, α-keto-glutarate).

α-Lipoic acid is used for the treatment of abnormal sensations associated with diabetic peripheral polyneuropathy, and for the treatment of disorders of the liver, and of poisonings by fungi and metals. Whereas the therapeutic effect was formerly often regarded as uncertain, recent dose-response studies and the establishment of a sufficiently high-dose therapy (>200 mg per day) in clinical use have improved the situation (summary of product characteristics for Thioctacid).

The biological and therapeutic effects of α-lipoic acid in oxidized and reduced form are also found in numerous derivatives and metabolites, sometimes in weakened and sometimes in improved form (for example in 3-ketolipoic acid, 1,2-diselenolane-3-pentanoic acid, lipoamide, octotiamine, 2-(NN-dimethylamine)ethylamidolipoate-HCl, tocopheryl lipoate and tocotrienyl lipoate, gamma-hydroxybuyrate/lipoate, lipoic acid/vitamin E ester, N-acetyl-p-aminophenol derivatives of lipoic acid and others) (Tirosch et al. 1999, see above, EP 0855 396 A1 EP 0869126 A1, PCT/GB98/02155, PCT/WO 99/06040, DE 4327 462 A1).

These derivatives were proposed in order to improve the metabolism and the distribution in vivo, which may also apply to distribution into the central nervous system. Some derivatives may also improve the effects (e.g. affinity and turnover rate) on the biological targets (biological redox systems such as α-keto-acid dehydrogenases, H-protein, thioredoxin, glutathione reductase or cellular redox systems such as glutathione, ubiquinone, complex I of the respiratory chain, or redox- and SH-sensitive proteins and enzymes, the NO system, catalase, the cellular cystine/cysteine shuttle, homocysteine, tyrosine kinase, MAP kinase, metal ions (for complex formation), alpha1-antiproteinase, or redox-sensitive transcription factors such as NF-kB or AP1) of α-lipoic acid, or couple other active molecules to α-lipoic acid with the aim of a synergistic or additive pharmacological effect.

For these reasons, the term “α-lipoic acid” is used in this text as a general term which, apart from the enatiomers, the racemate and mixtures of the enatiomers, also covers derivatives (esters, thioesters, ethers, salts, amides, metabolites etc.) as long as the active dithiolane group of α-lipoic acid is still partly responsible for the biological and medicinal effect of the derivative.

Because of the antioxidant function of α-lipoic acid, it has been proposed to employ α-lipoic acid as food supplement or product for medical nutrition alone or [lacuna] combination with other substances (U.S. Pat. No. 5,569,670, U.S. Pat. No. 5,599,835). Food supplements or medical foods are product categories controlled by particular national laws, which control the production and the marketing of products for particular health purposes in humans outside the conventional pharmaceutical sector.

α-Lipoic acid has further been proposed for the treatment of circulatory disorders in smokers and diabetics (U.S. Pat. No. 5,532,269) and as rheological agent in diabetics (DE 4439477C2).

It has been found that the R(+) enantiomer is advantageously suitable for controlling inflammatory disorders and for cytoprotection of cells (EP 0382066 A2).

The R(+) enantiomer has been proposed as an advantageous derivative of racemic α-lipoic acid for the treatment of insulin resistance (DE 4343593 Al) and for the treatment of disorders of glucose uptake in the central nervous system (DE 4343592C2).

The advantageous use of α-lipoic acid for disturbances of transmission in the central nervous system has been described (EP 0530446B1). Because of the free radical-trapping action, wide-ranging investigations which describe the neuroprotective effect of α-lipoic acid have been carried out. These include damage to the central nervous system associated with cerebral ischemia, damage through excitatory amino acids, mitochondrial damage, stated briefly damage to brain tissue in which free radical processes predominate (Packer et al., 1997, Loske et al., 1998).

In contrast to these wide-ranging investigations on protection of nerve cells in acute and chronic disorders of the central nervous system, to date only few data are available on the influencing of functional performance losses in the brain. Initial findings on the improvement of manifestations of dementia were described in patients with sclerotic dementia and in involutional depressions after administration of 25-50 mg of α-lipoic acid (Boysen 1967). However, these findings have not been confirmed in the decades of use of α-lipoic acid and must be regarded as misleading because of the, according to current knowledge, gross underdosage.

It has, however, subsequently been described that α-lipoic acid in high dose does in fact lead to an improvement in memory performance in aged rats but not in young animals (Stoll et al, 1993, Stoll et al, 1994). A beneficial influence on motor functions in aging animals has been described for R(+)-α-lipoic acid (Hagen et al., 1999).

A first clinical study was initiated on the basis of this rationale. However, no effect whatsoever was seen with effective-dose (600 mg) α-lipoic acid therapy on the cognitive function of patients with HIV dementia, although this had been expected because of the antioxidant properties of lipoic acid on the one hand, and of the numerous findings in animal experiments on the neuroprotective effect and on the improvement of learning in aged rats, and had formed the rationale for the clinical study. However, α-lipoic acid was not without effect; in fact, there was a deterioration in learning (Di Rocco et al. 1999, Dana Consortium 1998) through administration of α-lipoic acid. The verbal learning ability and the memory declined significantly. There were deteriorations in all the other neurophysiological tests applied. Thus, α-lipoic acid was not without effect; contrary to the rationale it had a marked pharmacological effect which caused the symptoms of dementia to deteriorate. This result is all the more disappointing because an active control drug (deprenyl) in this study confirmed its known beneficial effects on cognitive performance in demented patients and thus a chance negative finding to the detriment of α-lipoic acid can be ruled out.

Dementia is a general designation for the inability of a person to utilize his own mental capacities. Dementia resulting from a brain disorder usually has a chronic or progressive course with impairment of many higher cortical functions, including memory, thought, orientation, comprehension, calculation, learning ability, speech and judgement. The cognitive impairments are usually accompanied by a deterioration in emotional control, in social behavior or in motivation.

Criteria for dementia include:

Loss of intellectual abilities to such an extent that social and occupational abilities are impaired

Personality changes, including reduction in emotional control or drive, or cruder social behavior (also emotional lability, irritability, apathy)

Problems with routine work

Serious memory problems, e.g. disturbances of short-term memory and of long-term memory. The ability to recognize common factors and differences becomes less

Learning new information becomes more difficult

Incorrect irrational decisions leading to dangerous situations

Incipient impairment of speech and of understanding of speech (aphasia)

Difficulty with coordinating movements (apraxia)

Inability to recognize objects and people (agnosia)

Loss of the ability to calculate and to write.

The exact processes causing the disease are still not yet definitively explained. There is often a great similarity in the clinical picture, and the pathogenetic cause or the type of dementia can often be established only post mortem. In addition, there are evidently many mixed types in which a plurality of organic cerebral causes of the dementia occur side by side simultaneously (or sequentially). Irrespective of the primary organic cerebral cause, neuronal dysfunction is evident for all dementias. This dysfunction is characterized by a disturbance of signal transmission in the brain (disturbance of neurotransmission), with the decreasing synthesis or diminished excretion of various neurotransmitters (such as acetylcholine, dopamine and glutamate, serotonin and others) or diminished post-receptor effect playing a part which differs individually depending on the type of disease. This disturbance of neurotransmission determines the clinical picture of dementia with all the different organic cerebral causes. In the advanced state, the dysfunction may progress as far as neuronal cell death. Chronic inflammatory processes appear to be significant in some types of dementia, especially HIV dementia and Alzheimer's dementia (McArthur et al., 1999; McGeer and McGeer, 1997).

The clinical process of loss of cognitive function slowly progresses and usually affects people in middle age and older. Aging is the most important risk factor for the development of dementia (Payami et al., 1997).

The most important types of dementia are:

1. Dementia Associated with Alzheimer's Disease

Alzheimer's disease is a primarily degenerative cerebral dementia of unknown etiology but with characteristic neuropathological and neurochemical features. It usually has an insidious onset and develops slowly but continuously over years (two to three years, but occasionally more). The onset is rarely in middle age (Alzheimer's disease with presenile onset) but is frequently in advanced age (Alzheimer's disease with senile onset). In cases prior to age 65 to 70 it is possible relatively frequently to observe familialy similar cases with faster progression and principal symptoms of temporal and parietal damage, including dysphasia or dyspraxia. Cases with a later onset tend to progress more slowly and are characterized by a more general impairment of the higher cortical functions. Patients with Down's syndrome likewise show an analogous form of Alzheimer's disease.

Characteristic changes are found in the brain: a pronounced reduction in neuron populations, especially in the hippocampus, in the substantia innominata, the locus coeruleus, the temporal parietal and frontal cortex; occurrence of neurofibrillary tangles which consist of paired helical filaments of cytoskeletal proteins; neuritic (argentophilic) plaques which consist mainly of amyloid, with a clearly progressive development (but also plaques without amyloid) and granulovacuolar bodies. Neurochemical changes have likewise been found, for example a marked reduction in the enzyme choline acetyltransferase, in acetylcholine and other neurotransmitters and neuromodulators.

The suspected diagnosis of Alzheimer's disease is usually made solely on the basis of the clinical assessment of the symptoms. Besides the presence of dementia, an insidious onset with slow deterioration are characteristic. Whereas the onset can usually be definitively established only with difficulty, the realization that deficits are present may occur suddenly to third parties. Further criteria are: absence of clinical indications or specific findings on examination which indicate a systemic or cerebral disorder which may cause dementia (e.g. hypothyroidism, hypercalcemia, vitamin B12 deficiency, niacin deficiency, neurosyphilis, normal-pressure hydrocephalus, subdural hematoma); absence of a sudden apoplectic onset or neurological focal signs such as hemiparesis, loss of sensitivity, scotomas and disturbances of coordination supervene in the early phase of the disease).

2. Vascular Dementia:

Vascular (formerly arteriosclerotic) dementia, including multi-infarct dementia, differs from dementia associated with Alzheimer's disease through the onset, the clinical features and the course. Quite frequently there are transient ischemic attacks with short disturbances of consciousness, transient pareses or loss of vision in the history. The dementia may also follow a number of acute cerebral vascular events or, less often, a single stroke. This type of dementia, which usually has its onset at advanced age, may appear abruptly after a single ischemic episode or develop gradually. It is usually the result of an infarction of the brain as a result of a vascular disorder, including cerebral vascular hypertension. The infarctions are usually small but have cumulative effects.

Dementia is a precondition for the diagnosis. The cognitive impairment is usually non-uniform, so that loss of memory, intellectual impairment and neurological focal signs may occur. Insight and judgement may be retained relatively well. A sudden onset, a stepwise deterioration and neurological focal signs and symptoms increase the probability of the diagnosis. It can in some cases be confirmed only by computed tomography or eventually by neuropathological investigation. Additional features which occur are hypertension, carotid bruits, emotional lability with transient depressive mood, weeping or uncontrolled laughing and transient episodes of clouding of consciousness or delirium, often caused by further infarctions. The personality is usually retained relatively well but in a number of cases personality changes with apathy or disinhibition or an accentuation of previous personality traits such as egotism, paranoid attitudes or irritability may develop.

3. Dementia Associated with Pick's Disease

Progressive dementia with onset in middle age, usually between the ages of 50 and 60, characterized by early, slowly progressive changes in character and loss of social abilities. The disease leads to impairment of intellect, memory and speech functions with apathy, euphoria and occasionally also extrapyramidal phenomena. The neuropathological picture shows localized atrophy of the frontal and temporal lobes, but without neuritic plaques and neurofibrillary tangles beyond the normal extent for the age. Cases with early onset tend to have a malignant course. The social and behavioral peculiarities quite frequently start before obvious memory impairments. In contrast to Alzheimer's disease, frontal cerebral symptoms are more pronounced than temporal and parietal cerebral symptoms.

4. Dementia Associated with Parkinson's Disease

A dementia which develops during the course of a pre-existing Parkinson's disease (especially the severe forms). It has not to date been possible to describe any unambiguously characteristic clinical features. The dementia may be different from a dementia associated with Alzheimer's disease or from vascular dementia; however, there are also indications that simultaneous occurrence of one of these two forms of dementia together with Parkinson's disease is involved.

5. Lewy Body Dementia

This form of dementia is assumed in cases with fluctuating cognition (especially variations in attention and vigilance) and two of three of the following points:

visual, detailed hallucinations

parkinsonian symptoms

exceptional neuroleptic sensitivity in relation to the development of parkinsonism.

6. Dementia Associated with Disease Caused by the Human Immunodeficiency Virus (HIV)

A disorder which is characterized by cognitive impairments which satisfy the clinical criteria for the diagnosis of dementia in the absence of a simultaneously existing disease or disorder (apart from HIV infection) which might explain the clinical picture. Typical complaints in HIV dementia are of forgetfulness, slowness, poor concentration and difficulties in solving problems and in reading. Apathy, reduced spontaneity and social withdrawal are frequent. In a distinct minority of those affected, the disease may give the appearance of an affective disorder, of a psychosis or with seizures. Physical examination frequently reveals tremor, impairment of rapid repeated movements, disturbance of balance, ataxia, hypertonia, general hyperreflexia, frontal disinhibition phenomena and impairments of eye pursuit movements and saccadic eye movements. Children may show impaired CNS development caused by HIV, which is characterized by developmental retardation, hypertonia, microcephaly and calcification of the basal ganglia. A difference from adults is that the neurological involvement usually occurs without opportunistic infections and neoplasms.

Generally, but not exclusively, HIV dementia leads to severe, comprehensive dementia, to mutism and to death.

Neuropsychiological methods for measuring the severity of dementia

Standardized methods are developed for routine dementia screening, e.g. the mini-mental state examination (MMSE) or the Alzheimer's Disease Assessment Scale (ADAS). Further methods would be: GDS: Global Deterioriation Scale (Reisberg et al., 1982), FAST: Functional Assessment Staging (Reisberg, 1988).

Mini-Mental State Examination (MMSE)

The mini-mental state examination test was published by Marshall Folstein in 1975 and was originally intended as a test to differentiate cognitive functions (Folstein et al., 1975). Because the test is short, it is now used as screening test in studies for establishing inclusion/exclusion criteria. The test covers orientation, attention, short- and medium-term memory, word-finding, reading, writing, and in addition a command to perform an action must be obeyed. The tasks in the test are designed so that they can normally be solved without difficulty by people without cognitive impairment. The maximum score achievable is 30, and a score of less than 24 indicates a dementing disorder. The following levels are generally differentiated (Zec et al., 1992).

MMSE score more than 24=very mild,

20-23=mild,

10-19=moderate,

0-9=severe

Alzheimer's Disease Assessment Scale-Cognitive Subscale (ADAS-Cog)

A further test for establishing the neuropsychological status of a dementia patient is the “Alzheimer's Disease Assessment Scale-Cognitive Subscale (ADAS-Cog)” (Rosen et al., 1984). The Alzheimer Disease Assessment Scale was invented by W. G. Rosen, R. C. Mohs, 1988, Rosen and K. L. Davis, 1984, and the German version was produced by R. Ihl and G. Weyer, Beltz Test 1993. The ADAS was not originally intended to be a diagnostic instrument, but separates dementing people from healthy control subjects of the same age very well. The following criteria can be distinguished in the ADAS: cognitive area, noncogn. area, memory, orientation/praxis, motor function, depression, psychotic symptoms, concentration/cooperation, speech. In the version used here, speech is included in the cognitive area, and thus a max. minus score of 70 is possible. It was possible to show for people with Alzheimer-type dementia that the complete scale increases by 5 to 7 points on average over the course of 12 months, and by 14 points on average over the course of 24 months. The items in the cognitive part are most sensitive in this regard.

A categorization can be undertaken for the ADAS as for the MMSE too:

| | | |
|---|---|---|
| ADAS score | less than 23 = | very mild, |
| | 24-380 = | mild, |
| | 39-53 = | moderate, |
| | more than 54 = | severe. |

The therapy of dementias

All reactive ingredients currently available in the dementia sector (including the nootropics) unfortunately provide only marginal improvements in therapy for patients in relation to the symptomatic cognitive deficits. The medicines often provide an improvement only for a few months and must be selected carefully on the basis of the tolerability profile. Even a slowing of the progression of the loss of cognitive function must be regarded as a therapeutic advance. Only rarely do patients respond so well that there is even a temporary improvement in cognition, but this does not lead to permanent stoppage of the course of the disease.

It is common to all active ingredients that the disturbed neuronal signal transmission is improved again in a wide variety of ways. That said both for the active ingredients which improve cerebral blood flow (ginkgo extracts, cinnarizine, dihydroergotoxins, nicergoline, piracetam, pentoxyfilline, pyritinol, vinpocetine), improve the performance of nerve cells or protect nerve cells (calcium channel blockers, NMDA antagonists, memantines) or improve signal transmission in the brain in other ways (e.g. pro-acetylcholinergics such as, for example, Aricept (active ingredient: donepezil), Exolon (active ingredient: ENA 713), or rivastigmine).

Because of the immense demand for further effective, well-tolerated medicaments, there is a number of substances in clinical development (galantamine, propentofylline). Antiinflammatory agents (“non-steroidal antiinflammatory drugs” such as ibuprofen, acetylsalicylic acid or diclofenac) are also employed in addition. There is also investigation of whether substances such as vitamin E show a clinical effect.

In summary, it can be said that, unfortunately, as yet there are only a very few therapeutic approaches which, at best, have a temporarily stabilizing effect but do not provide permanent protection from further progression of the dementia.

It has now been found, surprisingly, that adjuvant administration of a-lipoic acid in sufficient dose to a basic therapy with dementia therapeutic agents (including neurotransmission enhancers) improves in a particular manner the ability deficits of demented people and retards or stops the natural deterioration in the deficit manifestations.

This behavior of a medicinal substance to show a clear and pronounced beneficial effect only in combination is unusual. Normally, a combination of two substances effective as monotherapy may have an additive or synergistic beneficial effect. An absent or even adverse effect of an active ingredient in monotherapy which then, however, is manifested as beneficial or is reversed into the beneficial in the combination is unexpected.

### EXAMPLES

Patients with clinical manifestations of dementing deficits and suspected brain damage based on Alzheimer's dementia or vascular dementia or a similar suspected diagnosis were treated with medicines to improve the symptomatic cognitive deficits (nootropics). Since all the active ingredients currently available in the dementia sector provide only marginal improvements, it was justified, despite the disappointing first study, to administer α-lipoic acid in high dose to patients on the basis of this basic therapy. The effect of the therapy was assessed using standardized clinical dementia assessment scales which are usual now and which are distinguished by high reliability. This comprises the mini-mental state examination (MMSE) or the Alzheimer's disease assessment scale (ADAS). The advantage of these clinical instruments is that the severity of the dementia can be established reliably, irrespective of the organic cerebral basis, which is usually not unambiguous, of the clinical picture of dementia.

Patients with Alzheimer's Dementia

### Example 1

Patient 1 (born in 1945), m., diagnosis of Alzheimer's dementia 12/98, at the start of the lipoic acid therapy: GDS: 3/4, moderate cognitive losses, FAST: diminished abilities to complete complex tasks, loses his work because the patient becomes very uncertain on his own, needs assistance in official dealings and in planning his daily work at home.

The patient has received Aricept (5/10 mg/day) for 4 months, during which the cognitive status deteriorated by 1 point in the MMSE. After 4 weeks of additional therapy with 600 mg of a-lipoic acid, the MMSE score improved by 2, and the ADAS score decreased by 6 to 18. The patient is again able to write his name and address legibly, to ride a bike and take part in sports.

### Example 2

Patient 2 (born in 1948) f, diagnosis of Alzheimer's dementia 10/97, at the start of the lipoic acid therapy: GDS: 5/6, moderately severe losses, FAST: needs a lot of help in performing daily tasks, must be looked after by her parents with whom she lives.

The patient has received Aricept (5/10 mg/day) for 12 months. The cognitive status has continuously deteriorated; in the MMSE score by 7 and in the ADAS score to 53.

Because of a depressive mood typical of dementia, the patient has been taken Remotiv (St. John's wort) for 5 months and 600 mg of vitamin E for 12 months. On taking up the additional therapy with 600 mg of lipoic acid, the patient's compliance improved considerably, and there was an improvement in the MMSE score of 1 point over the course of the next 7 months, and in the ADAS score by 11 points.

### Example 3

Patient 3 (born in 1930) m, diagnosis of Alzheimer's dementia 5/97, at the start of lipoic acid therapy: GDS: 4/5, moderate cognitive losses, FAST: needs a lot of help from his wife.

The patient has been treated with Aricept (5 mg/day) for 13 months and additionally with 600 mg of vitamin E. The MMSE score deteriorated by 2 in this time, and the ADAS score increased by 14. After additional therapy with 600 mg of lipoic acid, the cognitive functions improved by 1 in the MMSE score, the ADAS score recovered by 2. The patient feels better and is again able to accompany his wife quite frequently. He is able to make small purchases because the shop is directly adjacent to where he lives. The GDS improved to 4.

### Example 4

Patient 4 (born in 1940) m, diagnosis of Alzheimer's dementia 11/96, at the start of lipoic acid therapy: GDS: 3/4 moderate cognitive losses, loss of employment, FAST: needs help from his wife.

Treatment with Aricept for 19 months (5/10 mg/day) and for 20 months with 300 mg of aspirin and 600 mg of vitamin E. The cognitive ability showed a decrease of 5 in the MMSE score and of 9 in the ADAS score. After use of 600 mg of lipoic acid for 8 months, an improvement of 2 in the MMSE score and the ADAS score went back 3 points. The patient is again able to take part in conversations, engage in sport and carry out small household tasks.

### Example 5

Patient 5 (born in 1931) f, diagnosis of Alzheimer's dementia 5/1998, at the start of lipoic acid therapy: GDS: 4/5 moderate cognitive losses, FAST: needs help with all housework, washing, clothing. Treatment with Aricept (5/10 mg/day) for 24 months, further treatment with Accuzide 20 (blood pressure-lowering agent), with Akatinol and 600 mg of vitamin E. The patient showed a deterioration of 6 in the MMSE score despite this therapy. In the first 3 months after administration of 600 mg of lipoic acid, the patient's MMSE score improved by 1, and the GDS improved by one level, now level 4. The status is currently being maintained. The patient likewise shows improvement in coordinated motor functions and is thus able to ride a bike again when accompanied and to do some work in the garden.

### Example 6

Patient 6 (born in 1919) m, diagnosis of Alzheimer's dementia 9/1996, at the start of lipoic acid therapy: GDS: 4/5 moderate cognitive losses, FAST: needs help in all areas of practical living.

The patient has been treated with Aricept (5/10 mg/day) for 13 months and has been taking aspirin 100 for 5 years and Rökan (Gingko biloba) and 600 mg of vitamin e for 3 years. Despite this, the MMSE score showed a deterioration of 5 in the dementia. Since being treated with 600 mg of lipoic acid there has been no further deterioration, the ADAS score has lost 8. The patient is able to take part in many activities outside the house and the GDS improved to 4.

### Example 7

Patient 7 (born in 1939) m, diagnosis of Alzheimer's dementia 4/97, at the start of lipoic acid therapy: GDS: 4 cognitive losses so that the patient had to give up his managerial position, FAST: problems dealing with authorities and complex matters.

The patient has been treated with Aricept (5 mg/day) for 6 months and additionally with 600 mg of vitamin E. The dementia showed a deterioration of 6 in the MMSE score in this time. After administration of 600 mg of lipoic acid, the progression soon came to a stop and after two months there was a slight improvement in the MMSE score by 1, while the ADAS remains constant. The patient is able to look after himself with little help for several days while his wife had to be away. He is again able to make small purchases.

It emerged that therapy with α-lipoic acid can be continued successfully even if the basic therapy is withdrawn again on a trial basis.

### Example 8

Patient 8 (born in 1942) m, diagnosis of Alzheimer's dementia 6/99, at the start of lipoic acid therapy: GDS: 3, patient must give up his occupation because he has lost the ability to calculate. Treatment with Aricept (5/10 mg/day) for 8 weeks, treatment with 600 mg of lipoic acid for 4 weeks. At the start of the treatment with 600 mg of lipoic acid the MMSE score was 18 and is now 20; the ADAS score has declined by 3 to 35 error points.

Patients with Other Types of Dementia

### Example 9

Patient 9 (born in 1938) m, diagnosis of Lewy body dementia 7/1996; GDS: 5/6 severe cognitive impairments, FAST: needs assistance from his wife in all actions of daily life. The patient has been treated with Aricept 5 mg for 12 months, Madopar T 1×125/daily and Madopar Depot for the night. The patient reached an MMSE score of 17. After treatment with 600 mg of lipoic acid, there is an improvement of 3 in the MMSE score, the patient's speech improves so that he is again able to communicate important matters to his wife, the progression stops. The patient is better able to take part in many everyday activities.

### Example 10

Patient 10 (born in 1929) m, diagnosis of frontal cerebral atrophy 3/1999, GDS: 3, FAST: patient is slightly aggressive and thus it is very difficult for his wife to manage him. The patient has been treated with Exelon 6 mg/day for 9 months, during which time the MMSE score decreased by 5. After additional treatment with 600 mg of lipoic acid, it was possible to achieve an improvement of 1 in the MMSE score. The patient is again able to take walks on his own and take part in many everyday activities, e.g. ride a bicycle.

### Example 11

Patient 11 (born in 1925) m, diagnosis of dementia plus parkinsonism 11/96; GDS: 5, FAST: patient needs help from his wife in all daily activities.

Patient has been treated with Aricept 5/10 mg/day for 17 months, additionally with Madopar T (3×125 mg) since 6/1999 and additionally with 600 mg of lipoic acid for 4 months. It was possible to stop the progression observed up till then, and no deterioration is currently evident. The MMSE and ADAS scores have remained at the same level. The patient is again better able to take part in small everyday activities.

### Example 12

Patient 12 (born in 1935) f, diagnosis of frontal cerebral atrophy since 4/1966; GDS: 3, FAST: patient noticeably aggressive with, in addition, cognitive deficits and motor aphasia.

She has been treated with Zoloft 50 (psychoactive drugs), aspirin 100, Gingko biloba, Eunerpan (psychoactive drugs, discontinued) and for 8 weeks with Aricept (5/10 mg/day) and for 4 weeks with 600 mg of lipoic acid. The MMSE score has regressed in this short period from 16 to 18, and the ADAS score decreased by 5 from 41 points to 36 points. The patient's compliance increases markedly (aggressive phases), so that her husband is considerably better able to care for his wife at present.

### PHARMACEUTICAL EXAMPLES

α-Lipoic acid or derivatives can be produced in oral dosage forms (tablets or capsules with rapid or slowed release, solutions for drinking or suspensions) or parenterally intravenously or intramuscularly from ampoules or ready-to-use infusions according to the general prior art as described, for example, in the documents listed below.

EP 901211340.5, EP 0858 802 A2, EP 0318 891 A1, EP 0 560 092 B1, US 005650429A, US 005334612A, US 005569670A, US 9755433 A, PCT/US99/11178 and Japanese published specification (hei) 1-335772 (25.12.1989).

The products produced in this way can be put on the market labeled for the purpose of use for dementias, it being necessary to comply with appropriate national regulations for the instructions for use for medical staff and patients. The products may in this connection normally range in the legal framework from medicaments or, where appropriate, also from food supplements.

It is possible to use effective dosages of α-lipoic acid as adjuvant to a dementia therapy. The dementias may vary widely in the organic cerebral basis (Alzheimer's, Pick's dementia, vascular dementia, parkinsonian dementia and many others or else on the basis of the common mixed types).

In this connection, the basic therapy can consist in the administration of active ingredients for symptomatic therapy of cognitive deficits associated with dementias. The α-lipoic acid can be given as additional therapy in cases of mild, moderate or severe dementia to one or more dementia products—in particular neurotransmission enhancers—or else be give alone at times after pretreatment has taken place until a significant deterioration appears.

Suitable for therapy are also predementia patients with distinct chronic or temporary impairments of cognitive abilities or the activities of daily life or general impairment of tone. This group of patients is referred to in the literature as MCI patients (mildly cognitively impaired). The priority for predementia patients and patients with mild dementia who as yet show little that is clinically remarkable is for preventive aspects such as retention for as long as possible of the occupational and private capabilities and ability to work and avoidance for as long as possible of the need for care without these therapeutic aims not having significance for fully demented patients too.

During the α-lipoic acid administration it is also possible to give other products for the therapy of cognitive impairments (polytherapy) or integrate psychiatric procedures into the individual treatment policy. Further aims of the therapy, besides the actual improvement of cognitive functions, may be: improvement in the impaired motor abilities and the physical and mental tone, alleviation of concomitant depressive and psychiatric/psychotic symptoms, improvement in the patient's compliance for the actual basic therapy and for the non-medical individual therapy regimen, also improvement in the tolerability of the overall therapeutic regimen, reduction in the need for care and expenditure on care, improvement in the activities of daily life, where appropriate restoration of the ability to work. The latter points may also be important in the framework of a predementia treatment policy.

α-Lipoic acid can be given in oral or parenteral adminstration (i.v., i.m.), it being possible to employ for oral therapy solid or liquid rapid-release or slow-release pharmaceutical formulations.

Dosages of 100-2000 mg of α-lipoic acid or equivalent molar quantities of a derivative—the intact dithiolane structure being regarded as molar unit—can be given as daily dose, it being possible to give the daily dose as single dose or in divided doses distributed over the day, preferably 1-3 dosages. Preferably, 300-1200 mg of α-lipoic acid are given as daily dose, it being possible to give the daily dose as single dose or in 2-3 divided equivalent doses distributed over the day.

Derivatives of α-lipoic acid in reduced or oxidized form (e.g. salts, esters, thioesters, ethers, amides, metabolites) analogously can be employed as long as they are administered in a dose so that equivalent concentrations or biological effects on the target structures (biological redox systems) are achieved.

Optically dextrorotatory α-lipoic acid (R(+)-α-lipoic acid or R(−)dihydrolipoic acid) is preferably employed or derivatives [lacuna] α-lipoic acid or derivatives thereof can be administered in free or fixed combination with one or more active ingredients for improving cognitive impairments in effective combination.

It is possible to add vitamin C and derivatives, vitamin E and tocopherols, ubiquinone, vitamin B1-12, thiamine, riboflavin, pantothenic acid, niacin, biotin, inositol, β-carotene, zinc, magnesium, selenium, taurines, choline, N-acetylcysteine and derivatives, unsaturated fatty acids, essential fatty acids of the n-3 and n-6 series, gamma-linolenic acid and derivatives, aspirin and non-steroidal antiinflammatory drugs, L-carnitine and derivatives, St. John's wort, garlic preparations in free or fixed combination to the α-lipoic acid.

α-Lipoic acid and the basic active ingredients for the therapy of cognitive impairments can be processed and used in the form of the product categories medicaments, food supplements or medical foods.

1. The use of α-lipoic acid in reduced or oxidized form or derivatives thereof with intact dithiolane structure in the form of enantiomers or pharmaceutically acceptable salts, amides, esters, thioesters, ethers or metabolites for the adjuvant therapy of dementias.

2. The use as claimed in claim 1 of R(+)-α-lipoic acid in oxidized or reduced form as derivative of α-lipoic acid.

3. The use as claimed in claim 1 or 2, where there is additional use of one or more dementia therapeutic agents or one or more active ingredients for improving neurotransmission.

4. The use as claimed in any one of claims 1 to 3, where there is additional use of one or more of the following substances, selected from vitamin C and derivatives, vitamin E and tocopherols, ubiquinone, vitamin B1-12, thiamine, riboflavin, pantothenic acid, niacin, biotin, inositol, β-carotene, zinc, magnesium, selenium, taurines, choline, N-acetylcysteine and derivatives, unsaturated fatty acids, essential fatty acids of the n-3 and n-6 series, gamma-linolenic acid and derivatives, aspirin and non-steroidal anti-inflammatory drugs, L-carnitine and derivatives, St. John's wort and garlic preparations.

5. The use as claimed in any one of claims 1 to 4 for predementia patients.

6. The use as claimed in any of claims 1 to 4, where α-lipoic acid is given alone at times as adjuvant therapy to one or more dementia therapeutic agents after a pretreatment has taken place until a significant deterioration appears.

7. A composition comprising α-lipoic acid in reduced or oxidized form or derivatives thereof with intact dithiolane structure in the form of enantiomers or pharmaceutically acceptable salts, amides, esters, thioesters, ethers or metabolites and additionally one or more dementia therapeutic agents or one or more active ingredients for improving neurotransmission.

8. The composition as claimed in claim 7, additionally comprising one or more of the following substances, selected from vitamin C and derivatives, vitamin E and tocopherols, ubiquinone, vitamin B1-12, thiamine, riboflavin, pantothenic acid, niacin, biotin, inositol, β-carotene, zinc, magnesium, selenium, taurines, choline, N-acetylcysteine and derivatives, unsaturated fatty acids, essential fatty acids of the n-3 and n-6 series, gamma-linolenic acid and derivatives, aspirin and non-steroidal anti-inflammatory drugs, L-carnitine and derivatives, St. John's wort and garlic preparations.
